(19) United States
(12) Patent Application Publication
(10) Pub. No.: US 2010/0262174 A1
(43) Pub. Date: Oct. 14, 2010

(54) Microsurgery for Treatment of Glaucoma

(75) Inventors: David Sretavan, Tiburon, CA (US); Christopher Guild Keller, Ei Cerrito, CA (US)

Correspondence Address: FENWICK & WEST LLP, SILICON VALLEY CENTER, 801 CALIFORNIA STREET, MOUNTAIN VIEW, CA, 94041, US

(73) Assignees: The Regents of the University of California, Oakland, CA (US); Mynosys Cellular Devices, Inc., El Cerrito, CA (US)

(21) Appl. No.: 12/377,235

(22) PCT Filed: Aug. 13, 2007
(86) PCT No.: PCT/US07/75843

§ 371 Date: May 28, 2010

Related U.S. Application Data
(60) Provisional application No. 60/837,377, filed on Aug. 11, 2006.

(51) Int. Cl. A61B 17/32; A61N 1/30; A61M 37/00; A61B 19/00

(52) U.S. Cl. 606/170; 604/20; 604/500; 128/898

(57) ABSTRACT

Surgical procedures and devices are used for treating medical conditions in the eye such as glaucoma. In particular, access to Schlemm's canal in the eye is obtained by cutting through a portion of the sclera to expose the canal. This incision may be made using a fixture that guides a microknife to make a precise cut. A catheter containing a knife may then be placed within Schlemm's canal, and the knife can be actuated to a position outside the catheter to allow an incision to be made. This incision may be made through the wall of the canal and into the surrounding trabecular meshwork, thereby improving the drainage of the aqueous humor from the eye and lowering the intraocular pressure.

### CROSS REFERENCE TO RELATED APPLICATIONS

This application claims the benefit of U.S. Provisional Application No. 60/837,377, filed Aug. 11, 2006, which is incorporated by reference in its entirety.

### BACKGROUND

This invention relates generally to medical procedures and devices for performing microsurgery on an eye, for example, for treating medical conditions such as glaucoma.

Glaucoma is a chronic, progressive, and irreversible form of blinding eye disease that affects approximately 3 million people in the United States, and an estimated 60 million people are at risk of developing the disease. Worldwide, it is estimated that about 70 million individuals are afflicted with glaucoma, and over 300 million are at risk for developing glaucoma in their lifetime. The risk of developing the disease increases with age, so glaucoma is expected to increase in importance as the general population ages.

Glaucoma is very strongly associated with an elevated intraocular pressure, which through mechanisms involving the optic nerve causes the loss of nerve cells in the retina that mediate vision. The ciliary body in the eye produces a fluid called aqueous humor. FIG. 1 illustrates how the aqueous humor drains from the eye. The fluid finds its way into the anterior eye chamber and exits the eye through the trabecular meshwork, a cellular structure located at the angle formed by the cornea and the iris. Aqueous humor filtered through the trabecular meshwork is then collected in Schlemm's canal, a collecting duct that encircles the eye 360 degrees next to the trabecular meshwork. Fluid in Schlemm's canal eventually drains into the venous system. A balance in aqueous humor production and drainage dictates. intraocular pressure.

In glaucoma, it is thought that alterations in the architecture of the trabecular meshwork results in an increase in resistance to fluid flow and cause an elevation in intraocular pressure. The trabecular meshwork is composed of the inner uveal meshwork, the corneoscleral meshwork, and the juxtacanalicular tissue. The juxtacanalicular tissue lies immediately adjacent to the inner wall endothelium of Schlemm's canal. These layers of the trabecular meshwork are extremely thin. The human inner wall endothelium and the juxtacanalicular tissue are reported to be 2 micron and 10-20 microns thick, respectively. Fluid produced in the eye must filter through these structures to gain access into Schlemm's canal and make its way into the collecting channels and eventually the venous system.

It has been observed that cutting of the uveal meshwork does not change outflow resistance; however, slit openings made into the tissue consisting of the corneoseleral meshwork, juxtacanalicular tissue, and the inner wall of the canal greatly reduce outflow resistance. From measurements of outflow resistance, it is estimated that this subregion of the trabecular meshwork accounts for 75% of the normal resistance to outflow. it has further been found that 75% of the resistance between the anterior chamber and Schlemm's canal is located within 14 microns from the canal wall, and that 50% of the resistance was located within 7 microns from the canal. Accordingly, the site of resistance has been localized to the very thin but critical juxtacanalicular and inner canal wall regions. Glaucoma therapy must therefore address these tissues specifically and effectively.

Experimental studies in which pharmacological agents have been used to disrupt the trabecular meshwork also support the localization of the site of resistance to the juxtacanalicular tissue and the inner wall of Schlemm's canal. Intraocular application of Latrunculin B, a drug that disrupts the actin cytoskeleton of cells, or H-7, a protein kinase inhibitor, both cause an increase in outflow facility. Histological evidence reveals that the increase in outflow facility is likely due to disruption of the inner wall of Schlemm's canal, the expansion of space within the juxtacanalicular tissue, and the separation of the inner wall from the juxtacanalicular meshwork. These data indicate that the most direct means of improving fluid outflow from the eye is to increase filtration through the juxtacanalicular tissue and the canal inner wall. A definitive method that avoids long-term drug use and the accompanying toxicity is the precise, selective opening up of these tissues, while leaving the rest of the drainage system intact.

Although current surgical treatments for glaucoma are directed at increasing outflow from the eye, they are invasive procedures that remove substantial amounts of ocular tissue and do not specifically address only the juxtacanalicular tissue and the canal inner wall. Approaches include laser trabecular ablation using excimer lasers and various YAG lasers. Laser ablation frequently provides only temporary relief, as the holes created in the trabecular meshwork eventually fill in due to the wound healing process. Goniotomy is an invasive procedure in which a surgical knife is inserted through the cornea into the anterior chamber to physically disrupt the trabecular meshwork. Trabeculotomy is a procedure in which a surgical suture is threaded 360 degrees within Schlemm's canal and the suture drawn together to rip the entire trabecular meshwork. Although effective for pediatric glaucoma, goniotomy and trabeculotomy are not effective in adults, presumably due to the unintended wound healing response triggered due to the extensive tissue trauma that is induced.

The current gold-standard procedure for treating glaucoma is trabeculectomy, of which approximately 100,000 cases are performed each year in the United States. Trabeculectomy is a major invasive ophthalmologic surgical procedure in which a scleral flap is raised underneath the conjunctiva, and a block of tissue is removed to gain access into the anterior chamber. The opening that is created allows aqueous humor to drain into the space beneath the conjunctiva resulting in a fluid bleb and thus lowering intraocular pressure. Trabeculectomy requires a high level of surgical skill and curries with it significant morbidity and failure rates. Implanted drainage devices arc often needed after glaucoma surgery to maintain patency and fluid flow. However, these devices themselves have complications, such as blockage, infection, and device extrusion.

Recently developed arc forms of non-penetrating glaucoma surgery, which unlike in a trabeculectomy, do not open the anterior chamber and decompresses the eye. For example, Viscocanalostomy is a more recently described form of glaucoma surgery in which Schlemm's canal is cannulated and a viscoclastic substance is injected in an effort to dilate the collecting system and improve outflow. Another is non-penetrating trabeculectomy (NPT), in which the inner wall of Schlemm's canal is stripped without entering the anterior chamber. Both viscocanalostomy and NPT have lower complication rates but have also been shown to be less effective in lowering intraocular pressure compared to standard trabeculectomy. The mechanism by which viscocanalostomy improves outflow from the eye remains controversial, and the procedure can cause tears and damage at multiple sites in the drainage system. Given the way in which viscocanalostomy and NPT are performed and surgical instrumentation used, neither procedure allows the specific controlled microscale removal of the juxtacanalicular tissue and the inner canal wall endothelium.

Given the drawback of existing treatment procedures for glaucoma patients, improved techniques and devices fur performing them arc needed.

### SUMMARY

Embodiments of the invention provide surgical techniques for treating medical conditions in the eye such as glaucoma. The techniques described herein involve the selective removal or other means of modifying portions of the trabecular meshwork and the inner wall of Schlemm's canal. Embodiments of the invention also include microsurgical devices that facilitate access to Schlemm's canal and the precise micro-incision and/or other modification of the trabecular meshwork and Schlemm's canal tissue. Surgical procedures in accordance with embodiments of the invention are relatively short in duration compared to previous surgical procedures, and they can be accomplished reliably with average surgical skill.

Embodiments of the invention include a method for performing microsurgery on an eye, e.g., for the treatment of medical conditions such as glaucoma. A surgeon cuts a portion of a patient's sclera to create an opening that provides access to Schlemm's canal in the patient's eye. The surgeon then inserts a catheter into the Schlemm's canal, where the catheter includes a microknife for making an incision. Once the microknife in the catheter is located in the desired position for cutting, the surgeon uses the knife to cut through an inner wall of the Schlemm's canal and into a volume of a surrounding trabecular meshwork, or any portion thereof. When complete, the surgeon removes the catheter and microknife from Schlemm's canal, leaving an incision that assists in draining of the aqueous humor to lower intraocular pressure and thereby treat the patient's glaucoma.

Various devices and instruments may be used to perform procedures described herein. In one embodiment, a device for performing an incision to access Schlemm's canal comprises a fixture that is shaped and otherwise configured to be mounted over an eye. The device includes a curved knife mounted on a slidable member, which sits within a guiding slot formed in the fixture. The guiding slot is configured so that it allows the knife to move along a path designed to cut a flap in the sclera that exposes the eye's Schlemm's canal. In use, in one embodiment, an operator places the fixture over the eye, possibly securing the fixture in place using a vacuum pressure between the eye and fixture. The operator then slides the slidable member along the guiding slot, causing the knife to make an incision through the sclera and exposing Schlemm's canal.

In one embodiment, a device for performing an incision through an inner wall of the Schlemm's canal and into a volume of n surrounding trabecular meshwork comprises a fixture that is shaped and otherwise configured to be mounted over an eye. A rotatable member is coupled to the fixture and can turn around the fixture, where the rotation is approximately about the optical axis of the eye. The rotatable member is also coupled to a catheter to enable rotation of the catheter relative to the fixture. The catheter has a curved length and a cross section suitable for insertion into Schlemm's canal of the eye, and the catheter also includes a deployable knife located in the catheter. The deployable knife can be deployed into a cutting position by an actuator, which is operably coupled to the catheter. In use, in one embodiment, an operator places the fixture over the eye, possibly securing the fixture in place using a vacuum pressure between the eye and fixture. The operator then inserts the catheter into Schlemm's canal until the knife is in a desired location for cutting: The operator deploys the knife using the actuator and then performs an incision by moving the catheter and knife assembly through a length of the canal. Once the cut is performed, the operator retracts the knife, removes the catheter from the canal, and removes the device from the eye.

These techniques enable a surgeon to perform minimally invasive microsurgery within Schlemm's canal, which results in relatively low collateral damage to neighboring tissues as compared with previous treatment techniques, allowing for improved treatment of medical conditions such as glaucoma. The techniques described herein also provide a high level of control of the positioning and orientation of the cutting instrument for precise location of incisions within the lumen of Schlemm's canal. Some embodiments may also enable extremely precise application of pharmacologically active entities to cells that have been opened microsurgically or are in the vicinity of the procedure.

### BRIEF DESCRIPTION OF THE DRAWINGS

FIG. 1 is a cross section of an eye, showing the path in which aqueous humor to drains from the eye to lower the intraocular pressure.

FIG. 2 illustrates an eye and a hypothetical torus that defines a cut made for accessing Schlemm's canal, in accordance with an embodiment of the invention.

FIG. 3A is a cross section of an eye illustrating a cut made for accessing Schlemm's canal, in accordance with an embodiment of the invention. FIGS. 3B and 3C are enlarged views of Schlemm's canal of FIG. 3A before and after the cut, respectively.

FIG. 4 is a perspective view of a microsurgical instrument for making a cut for accessing Schlemm's canal, in accordance with an embodiment of the invention.

FIGS. 5A and 5B are front and side views, respectively, of the cutting blade of the microsurgical instrument of FIG. 4, in accordance with an embodiment of the invention.

FIG. 6 illustrates a catheter assembly for insertion into Schlemm's canal, in accordance with an embodiment of the invention.

FIG. 7 illustrates a cross section of the catheter of FIG. 6 at a location away from the end of the catheter, in accordance with an embodiment of the invention.

FIG. 8 illustrates a cross section of the catheter of FIG. 6 at a location near the end of the catheter, in accordance with an embodiment of the invention.

FIGS. 9A and 9B illustrate the actuation of a microknife by a ramp within a catheter, in accordance with an embodiment of the invention.

FIGS. 10A and 10B illustrate the flexure actuation of a microknife, in accordance with an embodiment of the invention.

FIG. 11 illustrates a catheter with a flexure-actuated curved microknife, in accordance with an embodiment of the invention.

FIGS. 12A and 12B show side and axial views, respectively, of a helical knife in accordance with one embodiment of the invention.

FIG. 13 is a cross sectional view of a catheter with a helical knife, in accordance with an embodiment of the invention.

FIG. 14 is a perspective view of a device for performing a micro-incision in Schlemm's canal and a portion of the surrounding trabecular meshwork, in accordance with is an embodiment of the invention.

FIG. 15 is a partial cutaway view of the device of FIG. 14.

FIGS. 16A through 16C show an alternative embodiment of a catheter that includes an integral cutting edge, in accordance with an embodiment of the invention.

FIG. 17 illustrates a method of cutting tissue using the catheter of FIGS. 16A through 16C, in accordance with an embodiment of the invention.

FIG. 18 shows an alternative embodiment of a catheter with a filament for advancing the catheter through Schlemm's canal, in accordance with an embodiment of the invention, and FIG. 19 is a view of the cutting edge of the catheter.

FIG. 20 is a perspective view of an embodiment of a catheter for performing is electrocauterization and delivering therapeutic agents, in accordance with an embodiment of the invention.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Overview of Treatment

Embodiments of the invention are described herein in the context of a medical treatment for glaucoma. This treatment involves making a precise cut into Schlemm's canal s to allow for a catheter to be placed therein. The catheter is equipped with a microknife, which can be actuated into a cutting position so that the knife can cut through the inner wall of Schlemm's canal and into the trabecular meshwork, or any fraction of that distance. The procedure may remove a volume of this tissue (e.g., on the order of 0.1 mm³ with a mass on the order of 0.1 mg), or it may be done so as not to remove any tissue, but simply make micro-incisions in the specific cells of interest and transfect them or other cells in the vicinity with appropriate genes or apply other agents or treatments. Once the desired treatment has been performed, the catheter is removed from Schlemm's canal and the initial access cut may be repaired. The result of the procedure is a very precise treatment of the trabecular meshwork for treating glaucoma effectively, without excessive damage to surrounding tissue Is where the treatment is not intended.

Embodiments of the invention include microsurgical instruments fabricated using microelectromechanical systems (MEMS) fabrication techniques, which produce devices on the order of 100-200 microns in size and have micron scale features that are well matched to the size of the fluid drainage systems of the eye. Due to the small size of these surgical devices, minimally invasive procedures can be used for their introduction into Schlemm's canal, thus enabling a surgeon to avoid the unnecessary dissection and handling of adjacent tissues (which is a source of the tissue scarring that leads to failure of other surgical methods). The small size of the incisions achieved by micro-fabricated cutting instruments also minimizes inflammatory responses in the trabecular meshwork, while maintaining the ability to target specific tissue layers for removal. Any inflammatory and wound healing responses may also be modified by the other functionalities of the device, including ideal gene transfection of meshwork cells, transplantation of exogenous cells, and the application of pharmacological and biological reagents.

### Exposing Schlemm's Canal

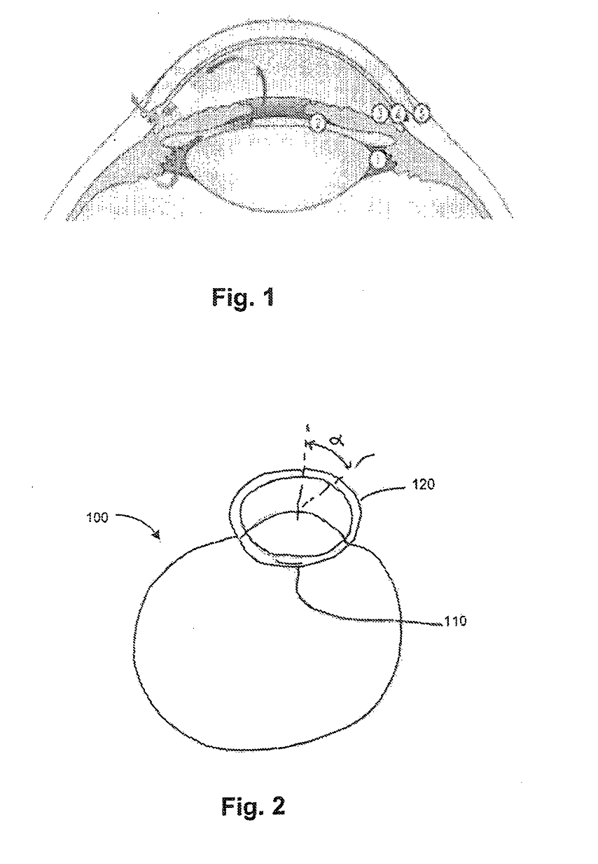

In one embodiment of a glaucoma treatment method, an initial incision is made for the purpose of gaining access to the Schlemm's canal of an eye to be treated. FIG. 2 illustrates a geometric description of the relationship between an eye 100 and the surface of a desired incision 110 into the eye 100. It will generally be desirable to minimize the disturbed area of the eye tissue while providing an opening to access Schlemm's canal, as well as to get tissue out of the way so that insertion of the catheter will not be obstructed.

As shown in FIG. 2, in one embodiment, the incision 110 is made at the intersection of the eye 100 and a torus 120. In this embodiment, the torus 120 has about the same dimensions as Schlemm's canal (e.g., a roughly elliptical cross section with a major radius of about 7 mm and a minor radius of about 0.5 mm). This torus 120 is located above Schlemm's canal but tilted at an angle so that part of the eye's sclera falls within the torus, thereby defining the site of the incision 110. In the diagram, the angle α is the angle between the axis of the torus 120 and the axis of Schlemm's canal.

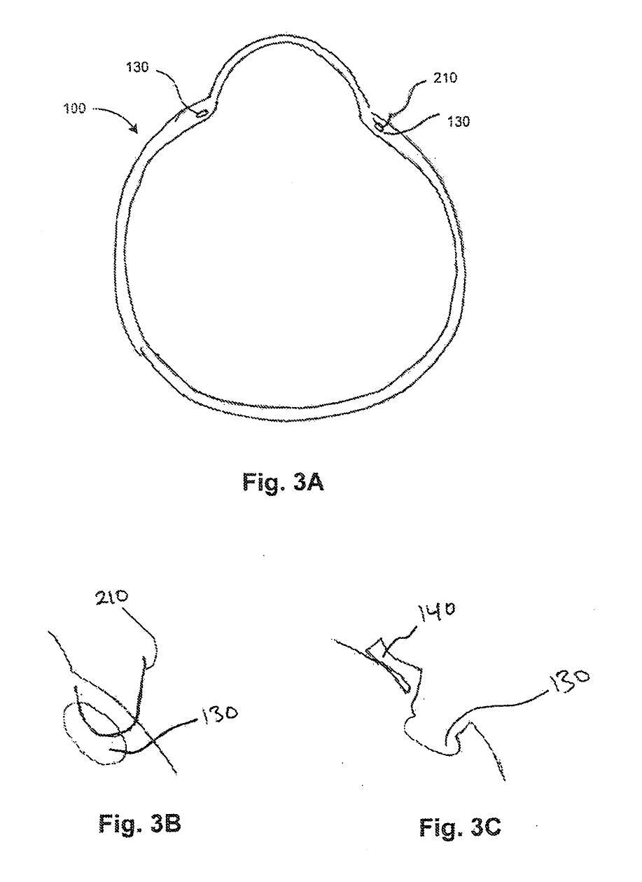

FIG. 3A shows a cross section of the eye 100, Schlemm's canal 130, and an axial view of a cutting blade 210. As will be described, the cutting blade 210 appears J-shaped from this cross sectional view, which leaves the cut tissue of the sclera attached to the eye 100 so that the cut flap of sclera can he replaced once the procedure is completed. FIG. 3B is a closer view of the blade 210 cutting an opening in Schlemm's canal 130. As shown in FIG. 3C, this cut leaves an uncut volume that allows the cut flap of sclera 140 to be rotated out of the way for catheter insertion into Schlemm's canal 130, and then placed back into its original position after the operation is over.

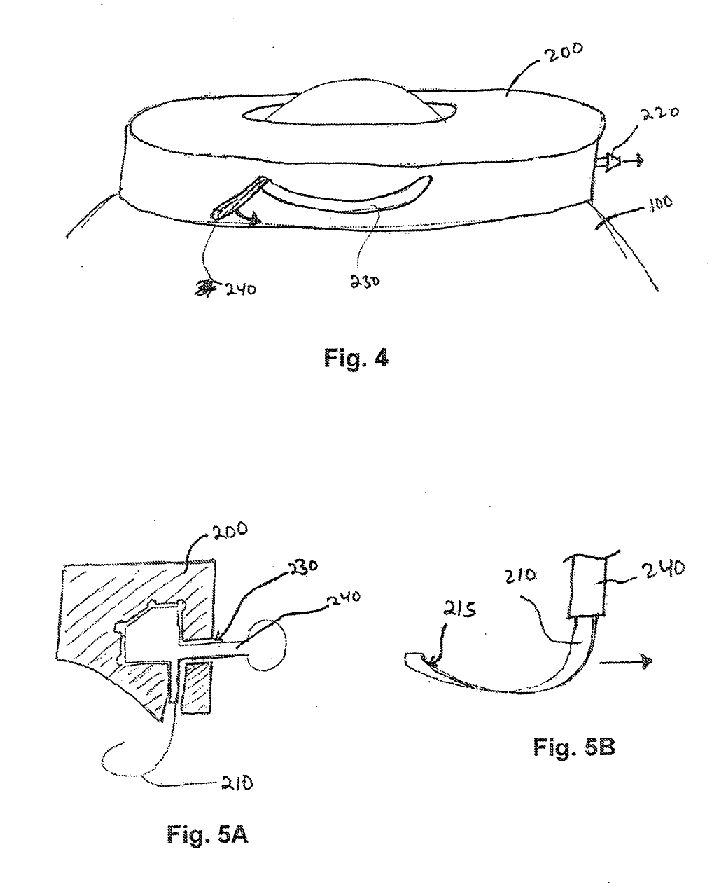

FIG. 4 illustrates an instrument for guiding the cutting blade 210 to make the incision 110 described above in a precise and predictable way. As illustrated, the instrument comprises a housing or fixture 200 that is configured to fit over an eye 100. When the instrument is fitted over the eye 100, in one embodiment, the housing 200 is held to the surface of the eye 100 by a vacuum. For this purpose, the instrument may comprise a vacuum port 220, which can be attached to a vacuum source for providing the vacuum pressure that secures the housing 200 to the eye 100. This vacuum pressure is maintained from when the instrument is placed in the desired location and orientation on the eye 100 to after the desired incision 110 is made.

The housing 200 also includes a guiding slot 230 and a slide member 240 that can slide along a path defined by the guiding slot 230. A cross section of the instrument where the guiding slot 230 runs through the housing 200 is shown in FIG. 5A. The slide member 240 is attached to the microknife 210, which appears as a J-shaped knife in the direction of cutting (also shown in FIGS. 3A and 3B). FIG. 5B is a side view of the cutting blade 210 and a portion of the sliding member 240. In this embodiment, the blade 210 is helical, which allows for a low angle of attack for the advancing cutting edge 215 to slice the tissue. The guiding slot 230 formed in the housing 200 constrains the movement of the sliding member 240 so that the blade 210 moves along a path that is part of a circular are that is inclined with respect to the axis of Schlemm's canal. This constraint produces an incision 210 that lies on the angled torus 120 illustrated in FIG. 2. In one embodiment, the structure is formed of disposable molded plastic components.

It is important that the housing 200 be designed and properly fitted to locate the blade so that it cuts the sclera at the correct radius and location of the eye 100 and penetrates to the correct depth. Although there are variations between individuals' eye geometries, the differences are small enough that one or just a few differently sized devices can cover the whole range of dimensions needed for treatment of human eyes.

During operation, in one embodiment, the housing 200 is placed over the eye 100 so that it is centered on the optic axis of the eye 100. The housing 200 is then fixed in place with respect to the eye 100 by activating a vacuum source coupled to the vacuum port 220. The sliding member 240 is then moved from one end of the guiding slot 230 to the other end in the direction of the cutting edge 215 of the microknife 210. This motion results in pulling the blade 210 through the sclera of the eye 100 to leave a cut whose surface is part of a torus, as shown in FIG. 2. The vacuum is then turned off and the fixture 200 is removed from the eye 100. An uncut flap 140 of tissue preferably remains to keep the cut portion attached to the eye 100 (sec FIG. 3C). This flap 140 can be lifted up (after the fixture 200 is removed) and held out of the way by surface tension forces against the cornea during the rest of the operation.

### Cutting Within Schlemm's Canal

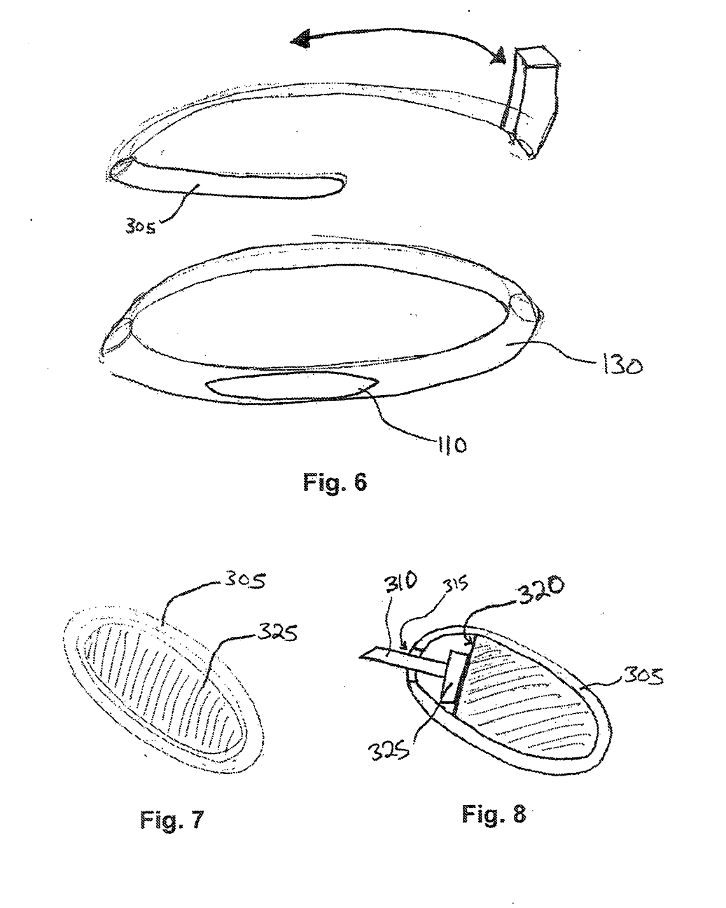

Once an incision is made to allow access into Schlemm's canal, a catheter 305 can then be inserted into Schlemm's canal 130. FIG. 6 illustrates a catheter 305, which in one embodiment is essentially a circular torus generated by the revolution of an ellipse whose major axis is inclined at an angle (e.g., the same angle found in Schlemm's canal, typically more than 45 degrees) towards the central axis. The size and shape of the catheter 305 are designed to match roughly the size and shape of Schlemm's canal 130, shown immediately below the catheter. In this way, when the catheter 305 is inserted into the lumen of the canal 130 through the incision 110, the catheter 305 occupies nearly the entire volume of the canal 130 and does not distort the natural shape of the canal 130. This avoids significant forces that might act to twist the catheter 305 or otherwise cause it to lose its orientation with respect to the direction of the trabecular meshwork, where cutting is intended.

FIG. 7 shows a cross section through the catheter at a location that is away from the end of the catheter 305 that is inserted into Schlemm's canal 130, and FIG. 8 shows a cross section through the catheter at a location that is near the end of the catheter 305 inserted into the canal 130. In FIG. 7, an inner member 325 is located within the catheter 305, and the inner member 325 and catheter 305 are slidable relative to each other. In one embodiment, the inner member 325 comprises silicon, and the catheter 305 comprises nickel.

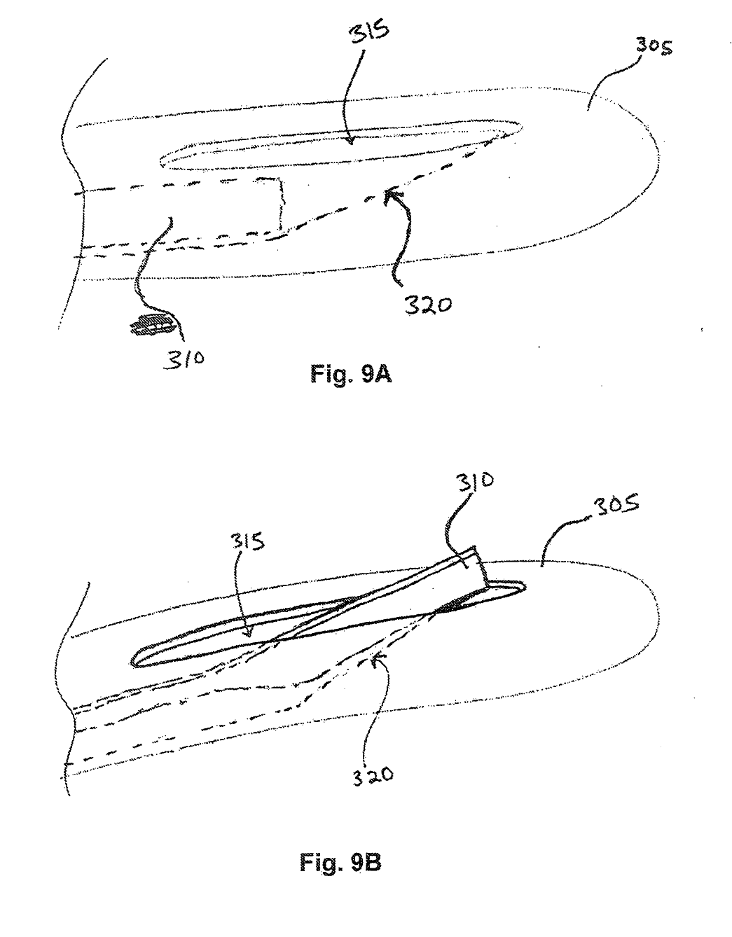

The inner member 325 is attached or otherwise mechanically coupled to a knife 310, which is located toward the end of the catheter 305, as shown in FIG. 8. Disposed at the end of the catheter 305 is a ramp 320, which guides the knife 310 outside of the catheter 305 through an opening 315 in the catheter 305, as shown in FIG. 8. The deployment of the knife 310 is further illustrated in FIGS. 9A and 913. In FIG. 9A, the knife 310 rests within the catheter 305, which position is useful when no cutting is desired (such as during placement of the catheter). In FIG. 9B, the deployment of the knife 310 is actuated from the opposite end of the catheter 305 by pushing the inner member 325 towards the knife 310. This causes the knife 310 to move towards the end of the catheter 305, and in combination with the ramp 320, it causes the knife to move partially outside the catheter 305 through the opening 315. To pull the knife 310 back within the catheter 305, the inner member 325 can be moved in the opposite direction, away from the knife 310, so that the knife 310 returns to the position shown in FIG. 9A.

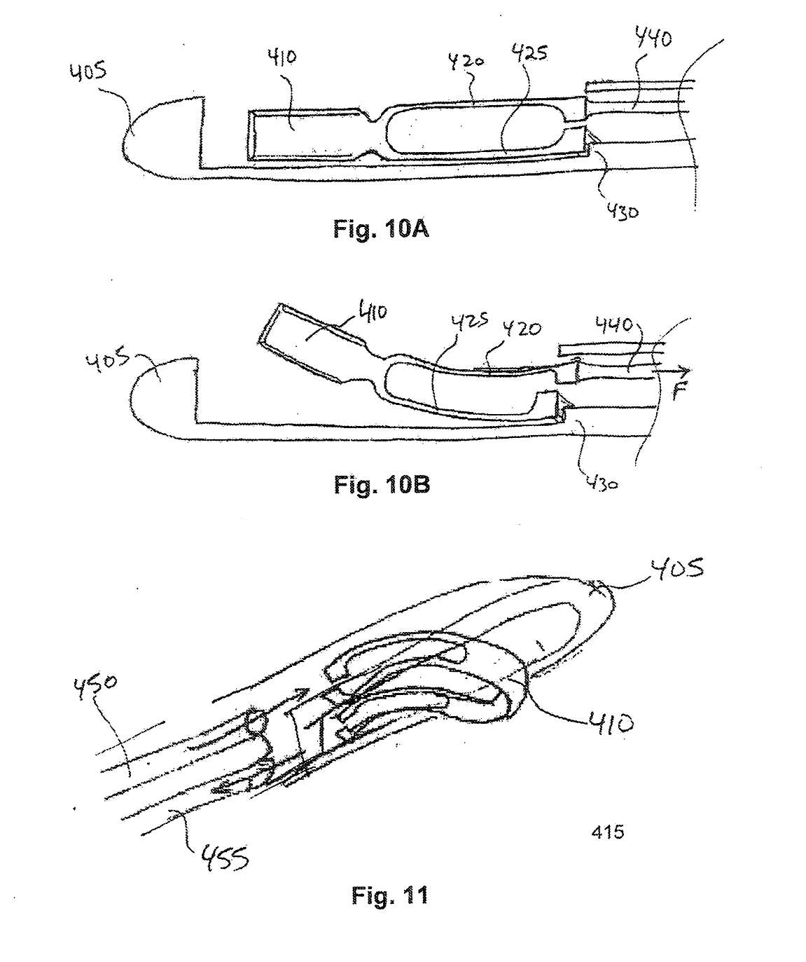

FIGS. 10A and 10B illustrate another embodiment in which a knife 410 is deployed by flexure. In this embodiment, rather than sliding the knife 310 up and down a ramp, the knife comprises two flexures 420 and 425. One flexure 425 sits against a stop 430 in the catheter 405, and the other flexure 420 is coupled to a pulling member 440. When a pulling force is applied to one flexure 420 by the pulling member 440, the result is to bend both of the flexures 420 and 425 so that the knife 410 deflects out of the catheter 405, as shown in FIG. 10B. With the knife 410 deflected in this way, the desired cutting can be performed. When the pulling force is removed, the knife then returns to its rest location within the catheter 405, as shown in FIG. 10A.

In one embodiment, the knife 415 is U-shaped or otherwise curved, as shown in FIG. 11. The curved shape of the knife 415 allows for a strip of tissue to be cut and removed, rather than merely making an incision. The curved knife may optionally have a piercing point (not shown) at the apex of the curve to facilitate in the cutting. Since the curved knife 415 allows for strips of tissue to be cut and removed, the catheter 405 may include an inflow channel 450 and/or an outflow channel 455. The inflow channel 450 allows water or other fluids to be introduced to the site where cutting is being performed, and the outflow channel 455 allows for removal of the tissue and any fluids introduced.

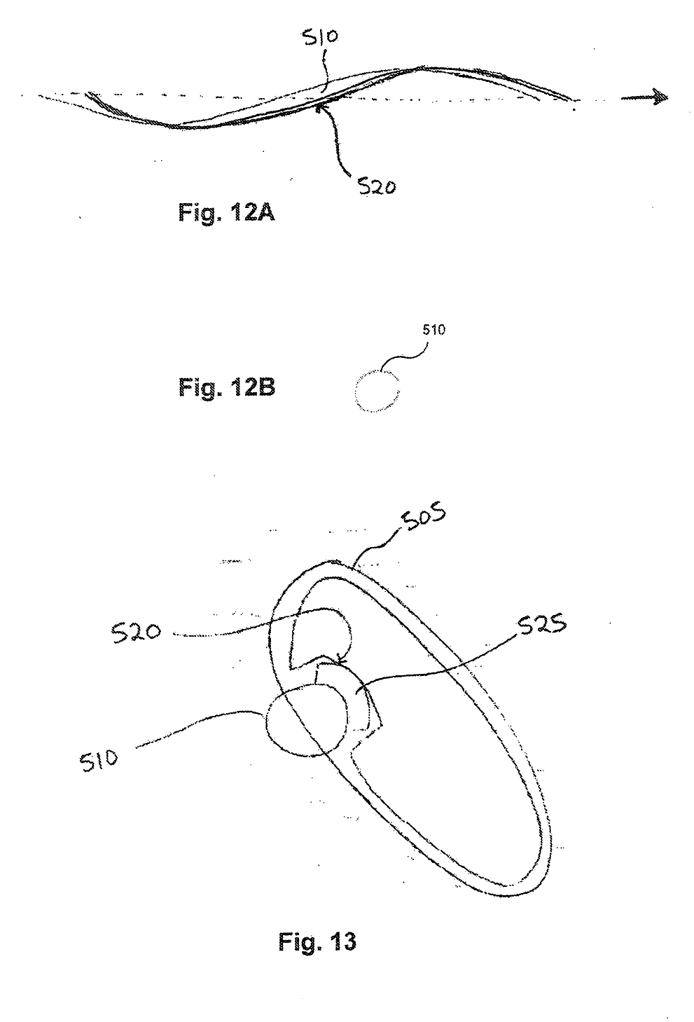

In another embodiment, a helical knife 510 is used as the cutting mechanism. FIGS. 12A and 12B show side and axial views, respectively, of a helical knife 510 in accordance with one embodiment. The helical geometry allows a continuous sharp edge 520 to confront a tissue surface at a low angle of approach, which means that the cutting force will be low. As seen from the axial projection, in FIG. 12B, the blade curves in a circle (or any other curved geometry, such as an ellipse). As the knife 510 is dragged through tissue it cuts a cylindrical surface, creating a partial cylindrical volume of tissue removed.

FIG. 13 is a cross sectional view of an end of a catheter 505 in which a helical knife 510 is used for cutting. Similar to the embodiment shown in FIGS. 7 through 9, the helical knife 510 can be deployed by pushing the knife 510 up a ramp 520 disposed within the catheter's end. This action forces the knife 510 outside the catheter 505 where it can cut,the wall of surrounding tissue, such as Schlemm's canal and the trabecular meshwork nearby. The knife is then retracted by pulling on a sliding member 525 to move the knife 510 down the ramp 520 and back within the catheter 505.

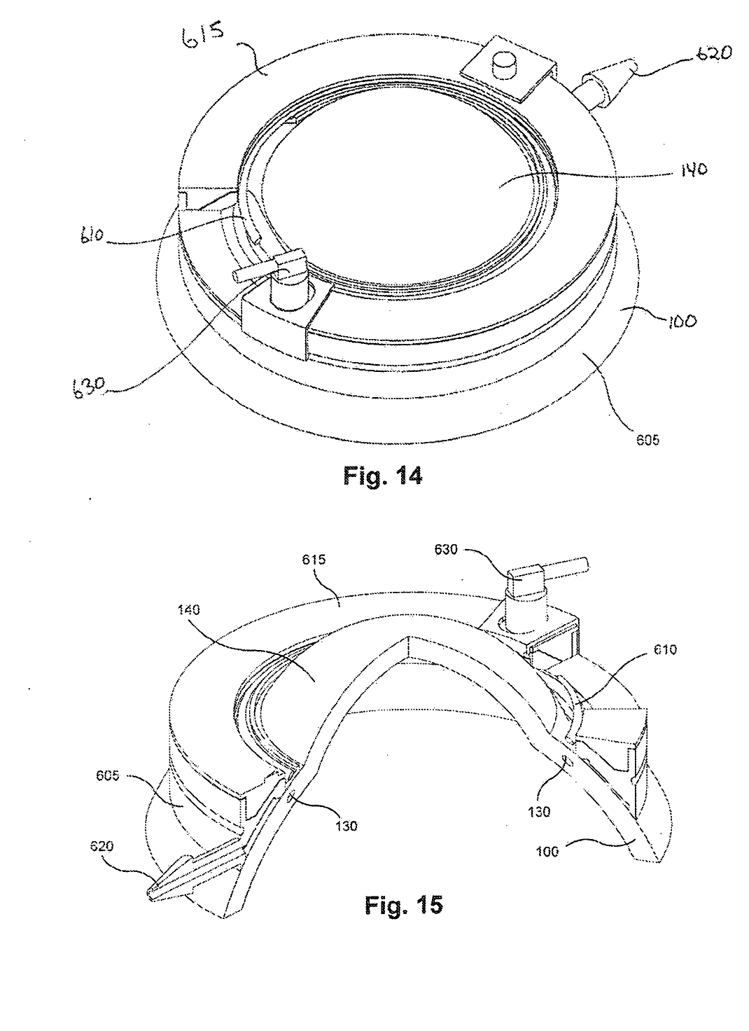

FIG. 14 is a perspective view of a device for performing a micro-incision into Schlemm's canal and a portion of the surrounding trabecular meshwork, and FIG. 15 is a cross sectional view of the same device, in accordance with an embodiment of the invention. As illustrated, the device is shown mounted on the surface of an eye 100. A base 605 of the device includes a vacuum port 620 where vacuum pressure can be applied to hold the base 605 in a position on the eye 100. In one embodiment, the base 605 only contacts the conjunctiva, which covers the sclera.

A rotatable ring 615 is mounted to the base 605 so that it can rotate around the base 605, and thereby rotate relative to the eye 100. The catheter 610 is fixed in relation to the rotatable ring 615 so that the catheter 610 rotates with the rotatable ring 615. The end of the catheter 610 is exposed by a cutout in the profile of the rotatable ring 615. The catheter is operably coupled to a actuator 630, which can be turned to deploy a knife within the catheter 610 to a location outside the catheter 610 for cutting into the surrounding tissue.

In one embodiment of the glaucoma treatment procedure, to make the desired incision through Schlemm's canal and into the trabecular meshwork, this device is centered on the optic axis of the eye. The device is then clamped down with vacuum force applied via the vacuum port 620. The surgeon will position the device so that the cutout in the rotatable ring 615 is aligned with the opening in Schlemm's canal 130. This cutout provides clearance for access to the opening in Schlemm's canal 130 that was created by the scleral flap cutting device described above. The rotatable ring 615 is moved so that the catheter 610 advances towards the opening that has been cut in Schlemm's canal 130. A small handheld tool (e.g., a hook) may be used to guide the end of the catheter 610 into the canal 130 as the ring 615 is related. Once the catheter 610 has entered the canal 130, the handheld hook is no longer needed, and the catheter 610 can be advanced further into the canal 130 by simply rotating the ring 615.

The rotatable ring 615 is rotated until the catheter 610 is in the desired location in Schlemm's canal 130. The end of the catheter 610 (and the knife therein) is known because the catheter 610 is fixed relative to the rotatable ring 615. Once the end of the catheter is at the desired location, the knife within the catheter is deployed using the actuator 630. The catheter 610 may comprise any of the embodiments described above, so the actuator 630 may deploy the knife in a number of different ways depending on the design of the catheter 610. For example, the actuator 630 may deploy the knife by moving a member to push the knife up a ramp, by pulling a member to cause flexure in the knife, or by any other actuation mechanism. This deployment drives the cutting edge of the knife into the tissue of the confronting inner wall of Schlemm's canal.

When the knife is in the deployed state, the catheter 610 can be withdrawn by rotating the rotatable ring 615 in the opposite direction. Because the knife is deployed into the surrounding tissue and is fixed within the catheter 610, this action causes the knife to make an incision through the canal 130 wall and into the trabecular meshwork for the distance that the ring 615 is rotated and the knife deployed. While the knife is deployed it will make a continuous cut; to make an intermittent cut, the knife can be retracted and redeployed as desired. An extremely precise incision is achieved because Schlemm's canal 130 itself is being used to guide the knit. Once the desired cut is made, the knit is placed back inside the catheter 610 using the actuator 630. The catheter 610 can then be withdrawn by rotating the ring 615 to the starting position. The scleral flap can then be replaced once the procedure is completed.

The catheter 610 may be constructed from various types of materials depending on the forces needed for the particular operation to be done. For operations needing high forces, for example, stiff materials having a high yield strength and a high modulus of elasticity may be used. In one embodiment, a high strength catheter 610 comprises an inner member of single crystal silicon enclosed by an electroformed nickel sheath. In one embodiment, an intermediate strength catheter 610 comprises a glass inner member enclosed by an electroformed nickel sheath. In one embodiment, a low strength catheter 610 comprises micromolded plastic inner and outer members.

Schlemm's canal is centered about the optical axis of the eye; therefore, when the device is placed on the eye, it should generally be centered about the optical axis as well. There are several ways to center the device, including mechanical alignment through contact with the side of the cornea, manual alignment with the operator looking through a low power microscope, and robotic alignment with a computer controlled manipulator and vision system.

As shown in FIGS. 14 and 15, there is a narrow gap between the structure of the device and the base of the cornea. To center the device on the cornea, an operator can see when the gap is of uniform width over the whole are of the structure (e.g., with the aid of a lens having a magnification of 3× to 5×). Once the device is in place, the operator can turn on the vacuum source to clamp the device in place. Generally, it is possible to position the device correctly within a tolerance of 0.1 mm, but even if the device is off by 0.5 mm the operation can still be performed successfully.

The forces that will be applied to the scleral flap cutting device and to the catheter device during the course of the operation will be small. Accordingly, a vacuum clamp will generally provide enough anchoring force to prevent the device from moving out of position. However, if stronger anchoring is desired, two or more small bumps or similar features'can be formed on the fixture to push against the sclera, locally deforming the eye to hinder sliding. These small bumps may he, for example, needle shaped and on the order of 0.25 to 0.5 mm long and 0.2 mm in diameter.

### Microknife

In one embodiment, any of the precise micro-incisions described herein can he made by a MEMS (microelectromechanical systems) microknife whose cutting edge has a radius of curvature on the order of nanometers. Such ultra-sharp cutting edges create minimal tissue tearing, distortion, and other tissue trauma, which activate an undesirable wound healing response. The “filling in” response of tissue observed alter more crude forms of tissue disruption can also be avoided.

In one embodiment, the microknives are self-sharpening, for example, as described in U.S. Provisional Application No. 60/837,401, filed Aug. 11, 2007, which is incorporated by reference in its entirety.

Catheter with Integrated Cutting Edge

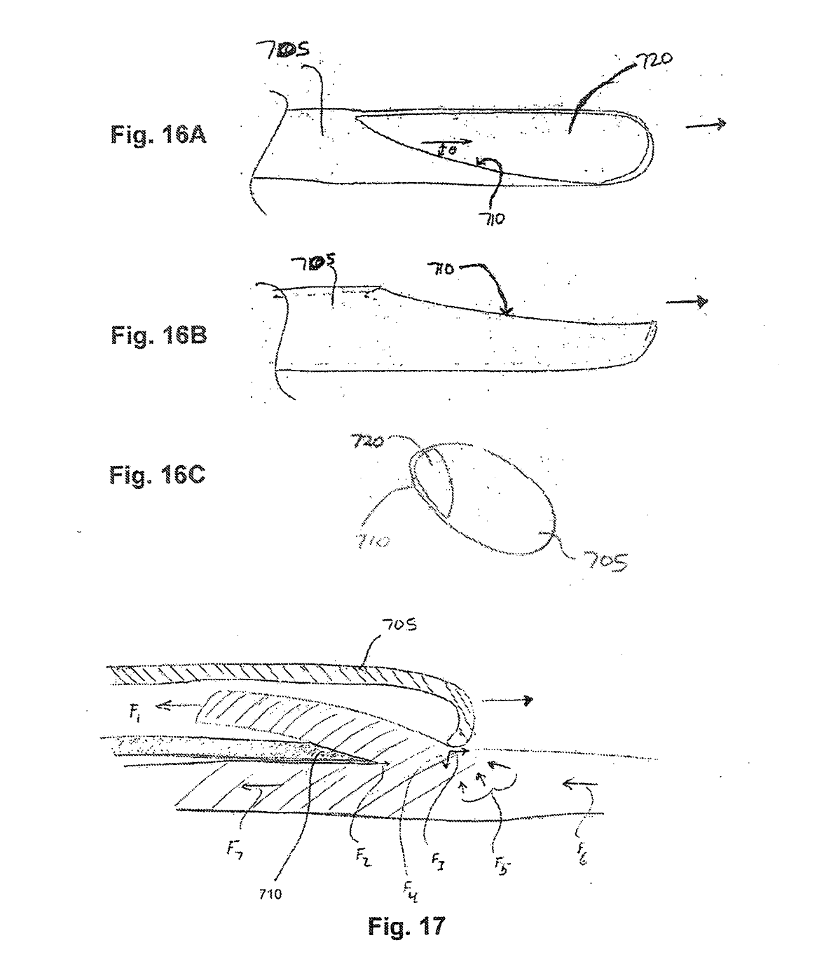

FIGS. 16A and 16B show top and side views, respectively, of a catheter 705 that includes a cutting edge 710 integrated into the wall of the catheter 705. The cutting edge 710 is formed with an opening 720 in the catheter 705. Preferably, the cutting edge 710 has an angle of attack θ that is small to make the cutting force small. FIG. 16C is an axial cross section view of the catheter 705 at the cutting end thereof. The catheter 705 has an elliptical cross section to match the cross section of Schlemm's canal, which helps to make the catheter 705 self-orienting in Schlemm's canal so the cutting edge 710 acts only on the area of the inner wall of the canal, or any part of the canal where cutting is desired.

FIG. 17 shows a cross sectional side view of the catheter 705 in use. Force vectors in the figure show the forces that act on the piece of tissue being cut. In one embodiment, a vacuum is applied from an opposite end of the catheter 705, causing a tensile force F₁ pulling on the tissue slice that is within the catheter 705. Outside atmospheric pressure exerts a force F₅, pushing tissue into the opening 720. Forces F₆ and F₇ in the surrounding tissue are reaction forces that arise in response to the forces applied by the catheter 705 (i.e., the cutting force F₂ and the friction forces F₃ and F₄).

Preferably, F₂, F₃, and F₄ are as small as possible, and F₁ and F₅ are large as possible. This facilitates the motion of the tissue slice past the cutting edge 710 and into the catheter 705. The cutting force F₂ is kept low by having a cutting edge 710 of near-atomic sharpness and oriented at a low angle of attack (i.e., low θ). Friction forces F₃, and F₄ may be reduced by coating the area of the catheter 705 that contacts the tissue with a lubricating material. The thin film of lubricating material may be any of the known low friction materials, such as Teflon. A greater tensile force (F₁) can be achieved if the catheter 705 is filled with degassed water. With degassed water, there is no dissolved gas to nucleate bubbles when tensile stress is applied (by pulling on a piston in a cylinder), so greater tension can be applied before the water molecules are pulled away from each other.

In use, the catheter 705 is inserted into Schlemm's canal through an opening created by making the scleral flap. No cutting occurs until a vacuum is applied to deform the tissue adjacent to the catheter opening. The reduced pressure causes a bulge of the inner wall of Schlemm's canal to form and extend into the catheter opening 710. As the catheter 705 is pushed forward, the cutting edge 710 slices through this bulge. When the vacuum is turned off, the tissue pulls back slightly, and the cutting edge 710 severs the connection between the cut strip of tissue and the wall of the canal. It is possible to make a continuous cut all the way around the canal, or to make intermittent cuts in any desired pattern by applying, or not applying, the vacuum while pushing the catheter forward. The cut pieces remain within the catheter 705 and are removed from Schlemm's canal when the catheter 705 is withdrawn.

### Push/Pull Catheter and Knife

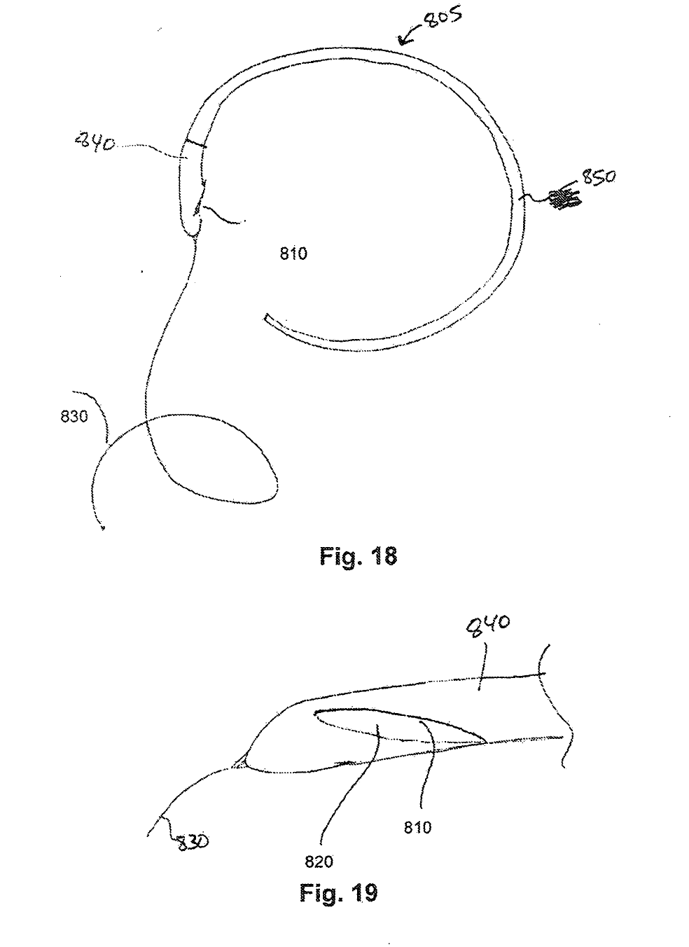

FIG. 18 illustrates another embodiment of a catheter 805 for making a cut through Schlemm's canal. The catheter includes a filament 830 that can be threaded through Schlemm's canal via a scleral flap opening (as described above). The filament 830 may be about 75 to 100 mm long to allow it to he threaded around the entire length of the canal. The filament 830 is connected to a rigid cutting head 840 of the catheter 805, which includes a cutting edge 810 and an opening 820. In one embodiment, the end of the thermoplastic filament 830 can be briefly melted, then cooled again, to form a smooth, round end to facilitate threading through the canal.

The cross section of the rigid head 840 is shaped like Schlemm's canal so that the cutting edge 810 will be properly oriented; however, the major and minor axes of the head 840 may be larger than Schlemm's canal so that the head 840 will stretch the tissue enough to force it into the knife. For example the major axis may be 400 microns and the minor axis 100 microns. The cutting edge 810 may also protrude from the cross section of the catheter 805 so that when it is pulled through the canal it cuts the surrounding tissue. Preferably, the cutting edge 810 confronts the inner wall of Schlemm's canal with a low attack angle for low-force cutting. When the cutting edge 810 cuts tissue, the cut strip of tissue enters the catheter 805 via the opening 820.

The orientation of the rigid head 840 is generally more stable the longer the head 840 is, but if the length comprises more than about 90° of the canal's are it will start to have too much drag through the canal. After the rigid head is a low, stiffness tube 850 designed to collect the cut tissue. The cross section dimensions of the tube 850 are smaller than Schlemm's canal to reduce frictional forces. The tube 850 can be open-ended or can terminate in a closed-ended bag.

The leading filament 830, the rigid head 840, and the trailing tissue collector tube 850 may be molded as one piece by injection molding of an appropriate polymer. Then, a silicon microknife may be attached to an edge of the opening 820 to form the cutting edge 810. Alternatively, the tube 850 can also be rigid so that it can be pushed around the canal at the base as well as pulled by the filament 830.

### Therapeutic Reagents

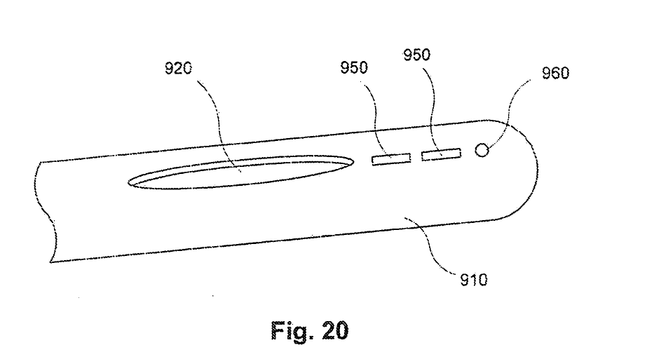

During cutting, in one embodiment, microfluidic ports in the microsurgical device permit the controlled release of desired phannacologic, biological, or tissue engineering reagents to affect the physiology, function, gene expression of the trabecular meshwork cells. The use of embedded electrodes in the catheter also allow the use of electroporation to enhance the entry of such reagents into the desired cells. FIG. 20, for example, illustrates a catheter 910 equipped with electrodes 950 for performing electrocauterization, as well as a fluid port 960 for delivery a therapeutic agent. The electrodes 950 and port 960, in one embodiment, are aligned with the opening 920 through which the microknife is designed to cut tissue, since that location is where an incision into tissue will be formed. The numbers of the electrodes 950 and port 960 and their locations may be chosen as needed, and various other configurations are possible to achieve the desired treatment.

The device can be used in conjunction with the delivery of pharmacologic and biologic reagents as well as genetic materials that will benefit the surgical outcome particularly in the magnitude, or duration, or stability of the reduction in intraocular pressure or the progression of disease. These reagents can be applied before, during, or after tissue cutting is performed.

The classes of pharmacologic, biologic, and genetic compounds that may be beneficial include those that affect tissue fibrosis such as mitomycin-C, and proteoglycans such as heparan-sulfate, compounds that affect the cellular cytoskeleton such as actin and microtubules, including cytochalasin and latrunculin, compounds that affect cellular signaling by interfering with kinase or phosphatase function, such as focal adhesion kinase, compounds that affect the assembly of extracellular matrix molecules such as laminin, fibronectin, including tissue proteases, and compounds that affect the function of junctional proteins such as adherens junctions. Also beneficial may be compounds that affect cell adhesion through effects on integrins, members of the immunoglobulin superfamily, cadherins, receptor tyrosine kinases such as the EphA and EphB families, and Eph ligands such as ephrins of the A & B subclasses. Also beneficial may be compounds that affect cell migration and recruitment such as chemokines and cytokines, vascular endothelial growth factors, and axon guidance molecules. Also beneficial may be compounds that affect the constituent function of lymphatic tissues such as podoplanin, LYVE-1, VEGFR-3, CCL21/SLC, LyP-1, Nrp2, integrin 9.1, Ang2/Tie2, CD31, CD34, PAL-E, D2-40, Desmoplakin, ICAM-1, VCAM-1, VWF. Also beneficial may be compounds that affect the inflammatory process and molecules activated during inflammation such as ELAM-1.

The use of genetic materials may involve application of viral vectors, such as Adenovirus, Aden-associated virus, and Lenti virus, to transduce the relevant cell, types in the trabecular meshwork and Schlemm's canal with dominant negative gene constructs that interfere with normal gene function, wild-type version of genes, or siRNA and anti-sense components that interfere with gene function.

### Alternative Embodiments

In one embodiment, the catheter includes electrodes for performing tissue and/or cell electrocauterization after the cutting. For example, as shown in FIG. 20, a catheter 910 may include electrodes 950 that are located in line with an opening 920 through which a microknife is designed to cut tissue. As the catheter is pulled through a lumen, the electrodes 950 pass across tissue that has been cut, so the electrodes can apply energy to cauterize the tissue to facilitate healing thereof. Alternatively, other energy sources can be used for cauterization, such as RF energy or laser energy sources.

Although one method of use of the invention involves cutting with a MEMS microknife, other embodiments of the invention may be employed in situations in which a microknife is not used, where other features built into the terminal structure are used to effect treatment for glaucoma.

The sequence of operations described above is one example method of use. The specific steps to be performed can be selected and the order of their performance can be rearranged. For example, cutting may he performed after drug delivery.

Although the dimensions of the device have been chosen in order to provide optimal use within the eye and for glaucoma surgery, the fundamental device design will have utility for other types of microsurgery in the eye or elsewhere in the body. The device also includes other non-medical uses, where access to small spaces must be combined with precisely controlled cutting, sampling, or other modifications of the accessed structures.

Embodiments of the invention have been described herein in the context of a microsurgical treatment for glaucoma. However, it will be appreciated that certain of the techniques and devices described may be used to achieve the same cuts or other microsurgical effects for treatment of other medical conditions. For example, the instruments and techniques described for making a cut to gain access to Schlemm's canal may be used for purposes other than to make a further cut into the trabecular meshwork for treatment of glaucoma. In addition, different methods for cutting to allow catheter insertion into Schlemm's canal may be used in combination with the instruments and techniques described herein for cutting into the trabecular meshwork for treatment of glaucoma.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for performing microsurgery on an eye, the method comprising:
cutting a portion of the eye's sclera to expose a Schlemm's canal of the eye;
inserting a catheter into the Schlemm's canal, the catheter including a microknife;
deploying the microknife into a cutting position;
cutting with the microknife through an inner wall of the Schlemm's canal and into a volume of a surrounding trabecular meshwork; and
removing the catheter and microknife from the Schlemm's canal.

2. The method of claim 1, wherein cutting a portion of the sclera comprises:
leaving the cut portion of the sclera attached to the eye.

3. The method of claim 1, wherein cutting a portion of the sclera comprises:
making an incision in the eye that approximates an intersection of the eye with a torus tilted with respect to the Schlemm's canal.

4. The method of claim 1, wherein cutting a portion of the sclera comprises:
fixing a cutting instrument over the eye, the cutting instrument including the microknife mounted on a member slidably mounted within a guide in the instrument;
sliding the microknife along the guide to cause an incision through the sclera and exposing Schlemm's canal.

5. The method of claim 4, wherein cutting a portion of the sclera further comprises:
fixing the instrument to the eye using a vacuum pressure between the instrument and the eye.

6. The method of claim 1, wherein the portion of the sclera is cut with a helical blade.

7. The method of claim 1, wherein the portion of the sclera is cut with a blade having a cutting edge with a radius of curvature that is less than 50 Angstroms.

8. The method of claim 1, wherein the portion of the sclera is cut with a blade that is self-sharpening.

9. The method of claim 1, wherein cutting into the Schlemm's canal comprises:
moving the catheter in the Schlemm's canal with the microknife deployed in the cutting position.

10. The method of claim 1, wherein deploying the microknife comprises:
pushing the microknife towards a distal end of the catheter, the distal end including a ramp and an opening, wherein the ramp guides the microknife outside the catheter through the opening.

11. The method of claim 1, wherein deploying the microknife comprises:
fixing a first flexure member of the microknife against a stop in the catheter;
pulling on a second flexure member of the microknife away from a distal end of the catheter, wherein the first and second flexure members bend to cause the microknife to move outside the catheter through an opening at the distal end thereof.

12. The method of claim 1, wherein the catheter has cross section substantially similar to the Schlemm's canal.

13. The method of claim 1, wherein the microknife is helical.

14. The method of claim 1, wherein the microknife has a cutting edge with a radius of curvature that is less than 50 Angstroms.

15. The method of claim 1, wherein the microknife is self-sharpening.

16. The method of claim 1, wherein the microknife is curved, and cutting with the microknife removes a strip of tissue.

17. The method of claim 16, further comprising:
collecting the cut strip of tissue in the catheter; and
removing the cut strip of tissue from the eye using the catheter.

18. The method of claim 1, further comprising:
adding a therapeutic agent via the catheter to at least a portion of the tissue cut with the microknife.

19. The method of claim 18, further comprising:
performing electroporation using electrodes on the catheter to enhance the entry of the therapeutic agent.

20. The method of claim 1, further comprising:
electrocauterizing at least a portion of the tissue cut with the microknife using electrodes on the catheter.

21. A device for performing microsurgery on an eye, the device comprising:
a fixture configured to be mounted over an eye;
a catheter having a curved length and a cross section suitable for insertion into a Schlemm's canal of the eye, the catheter including a deployable knife located therein;
a rotatable member rotatably coupled to the fixture, the rotatable member further coupled to the catheter to enable rotation of the catheter relative to the fixture, the rotation being approximately about an optical axis of the eye; and
an actuator operably coupled to the catheter for deploying the knife into a cutting position.

22. The device of claim 21, wherein the catheter comprises a ramp and an opening at a distal end thereof, the ramp configured to guide the microknife outside the catheter through the opening when the-knife is deployed.

23. The device of claim 21, wherein the microknife comprises a first flexure member and a second flexure member, and the actuator is operably coupled to pull on the first flexure member for causing the microknife to bend to move outside the catheter through an opening at the distal end thereof.

24. The device of claim 21, wherein the catheter has cross section substantially similar to the Schlemm's canal.

25. The device of claim 21, wherein the microknife is helical.

26. The device of claim 21, wherein the microknife has a cutting edge with a radius of curvature that is less than 50 Angstroms.

27. The device of claim 21, wherein the microknife is self-sharpening.

28. The device of claim 21, wherein the microknife is curved for removing a strip of tissue.

29. The device of claim 21, wherein the catheter includes a port for delivering a therapeutic agent to at least a portion of the tissue cut with the microknife.

30. The device of claim 29, wherein the catheter includes one or more electrodes for performing electroporation to enhance the entry of the therapeutic agent.

31. The device of claim 21, wherein the catheter includes one or more electrodes for electrocauterizing at least a portion of the tissue cut with the microknife.

32. A device for performing microsurgery on an eye, the device comprising:
a catheter including a microknife deployable into a cutting position;
means for locating the catheter in a Schlemm's canal of the eye; and
means for deploying the catheter to cut through the inner wall of the Schlemm's canal and into a volume of a surrounding trabecular meshwork.

33. The device of claim 32, further comprising:
means for making an incision to expose Schlemm's canal.

34. A device for performing microsurgery on an eye, the device comprising:
a fixture configured to he mounted over an eye, the fixture including a guiding slot;
a slidable member configured to fit within and move along the guiding slot; and
s a microknife attached to the slidable member,
wherein the guiding slot is oriented to guide the microknife to cut into a Schlemm's canal of the eye when the fixture is mounted on the dye.

35. The device of claim 34, wherein the microknife is curved, and the guiding slot is oriented to guide the microknife to leave the cut portion of the sclera attached to the eye.

36. The device of claim 34, wherein the guiding slot is oriented to guide the microknife to make an incision in the eye that approximates an intersection of the eye with a torus tilted with respect to the Schlemm's canal.

37. The device of claim 34, wherein the device further comprises:
a vacuum port for coupling a vacuum source to the device for creating a vacuum pressure between the fixture and an eye.

38. The device of claim 34, wherein the microknife comprises a helical blade.

39. The device of claim 34, wherein the microknife comprises a blade having a cutting edge with a radius of curvature that is less than 50 Angstroms.

40. The device of claim 34, wherein the microknife comprises a blade that is self-sharpening.
